(19) United States
(12) Plant Patent Application Publication
(10) Pub. No.: US 2013/0347150 P1
(43) Pub. Date: Dec. 26, 2013

(54) Blueberry plant named 'Blue Silk'

(50) ;

(71) Applicants: Jessica Scalzo, Havelock North (NZ); Narandra Patel, Hamilton (NZ)

(72) Inventors: Jessica Scalzo, Havelock North (NZ); Narandra Patel, Hamilton (NZ)

(21) Appl. No.: 13/815,545

(22) Filed: Mar. 8, 2013

Related U.S. Application Data
(60) Provisional application No. 61/664,156, filed on Jun. 26, 2012.

(51) Int. Cl. A01H 5/00

(52) U.S. Cl. PLT/157

(57) ABSTRACT

A new and distinct blueberry variety is described. The variety results from selection among a population of seedlings derived from the controlled cross of the varieties ‘Elliott’ (not patented) and ‘Puru’ (U.S. Plant Pat. No. 6,701). The new variety is distinguished from others by its late seasonality, fruit size, and plant habit. Fruit of ‘Blue Silk’ is medium-large in size with a medium-strong bloom.

### GENUS AND SPECIES OF PLANT CLAIMED

Vaccinium corymbosum

### VARIETY DENOMINATION

‘Blue Silk’

### CROSS REFERENCE TO RELATED APPLICATION

This application claims the benefit of U.S. Provisional Application No. 61/664,156 filed Jun. 26, 2012, the entire disclosure of which is incorporated by reference in its entirety.

### BACKGROUND TO THE INVENTION

Seeds obtained from the deliberate crossing of the varieties ‘Elliott’ (not patented) and ‘Puru’ (U.S. Plant Pat. No. 6,701), which occurred at Ruakura, Hamilton, New Zealand were shipped to Gilten, Germany. Seedlings obtained from this cross were planted out in Gilten, Germany in 2002. Three seedlings were selected from this family in 2004, one of which was given the breeder code ‘ZDM035’. In the Northern Hemisphere summer of 2008 ‘ZDM035’ was identified as having potential as a new variety and in 2009 it was asexually propagated. The resulting plants were planted out at Gilten and were subsequently found to be true to type demonstrating that the characteristics of the new variety are stable and transmitted without change through succeeding generations. Propagation has since been carried out through both soft and hard wood cuttings. ‘ZDM035’ was later designated ‘Blue Silk’.

### SUMMARY OF THE INVENTION

‘Blue Silk’ is characterised by its late seasonality, fruit size, bloom, and upright habit. ‘Blue Silk’ is distinguished from a number of other varieties by the following characteristics:

‘Blue Silk’ is harvested significantly later than ‘Bluecrop’ (not patented) grown in Gilten, Germany. Fruit of ‘Blue Silk’ have been observed to be starting to turn blue when approximately 50% of the ‘Bluecrop’ crop has been harvested.

‘Blue Silk’ produces medium to large fruit, averaging 2.2 g, whereas ‘Nui’ (U.S. Plant Pat. No. 6,699) produces large fruit, averaging 3 g, which reach harvest maturity earlier, under the same conditions in Germany.

### BRIEF DESCRIPTION OF THE DRAWINGS

FIG. 1 shows a mature, six year old ‘Blue Silk’ plant.

FIG. 2 shows a juvenile, two year old plant.

FIGS. 3 and 4 show show fruit of ‘Blue Silk’ on the plant.

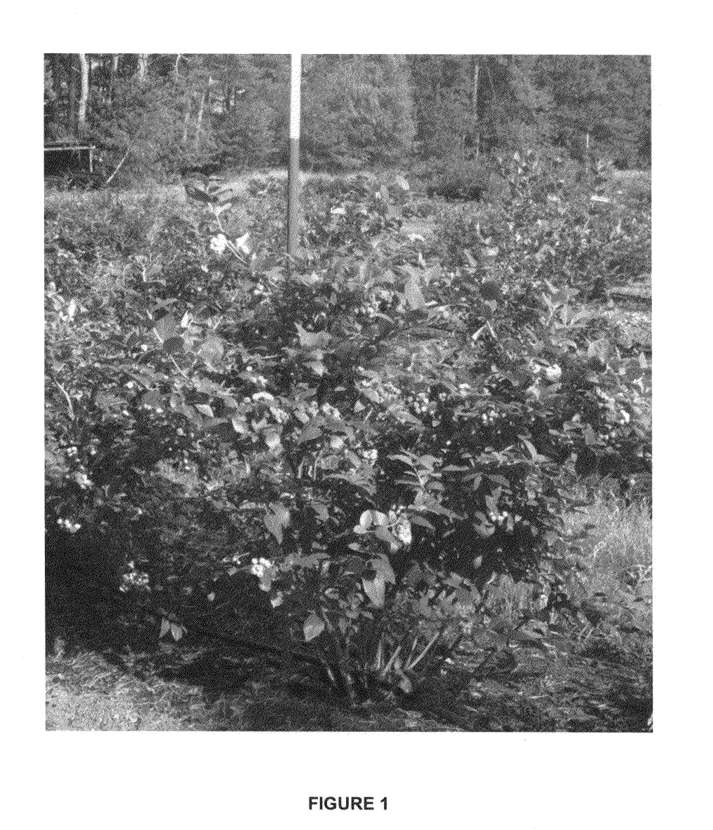

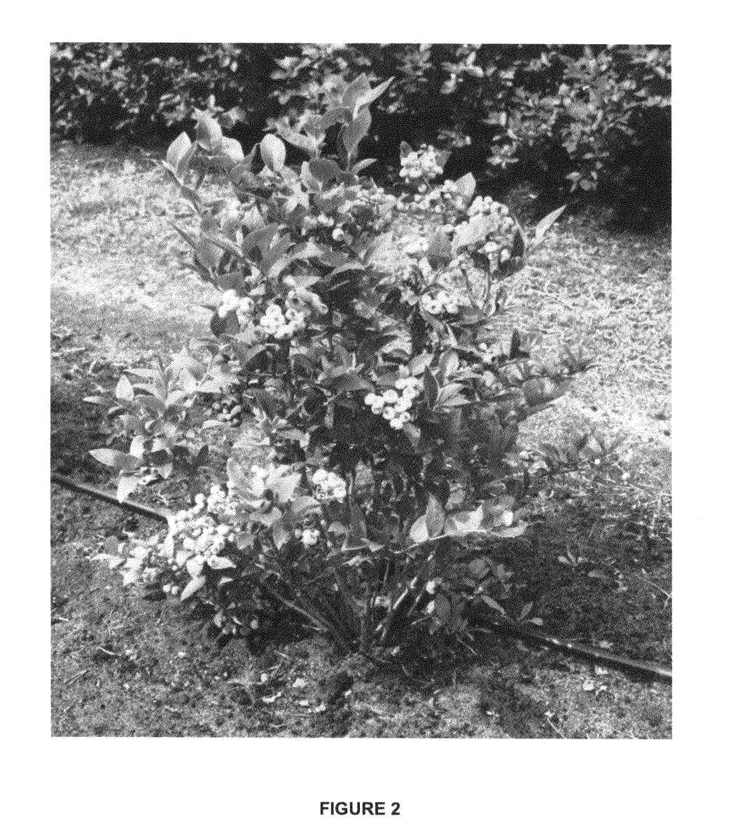

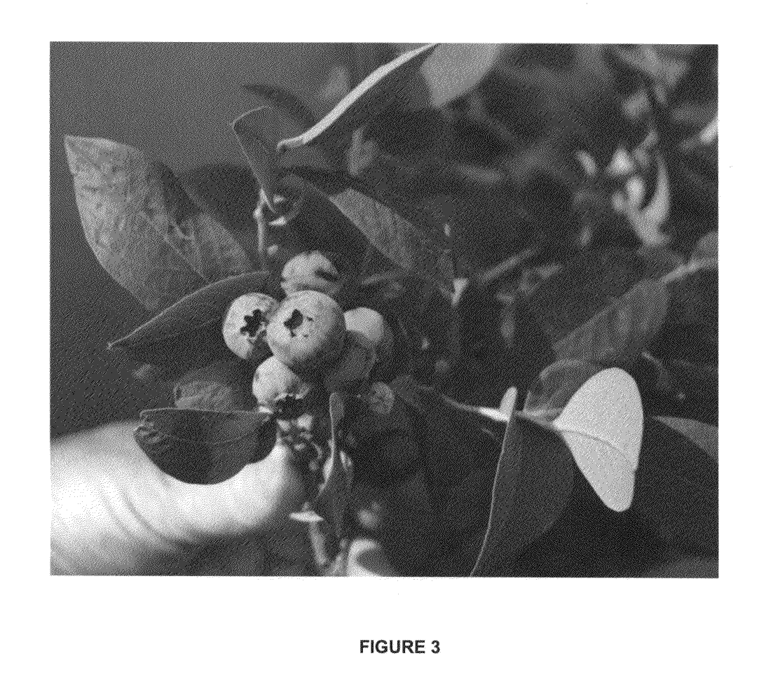

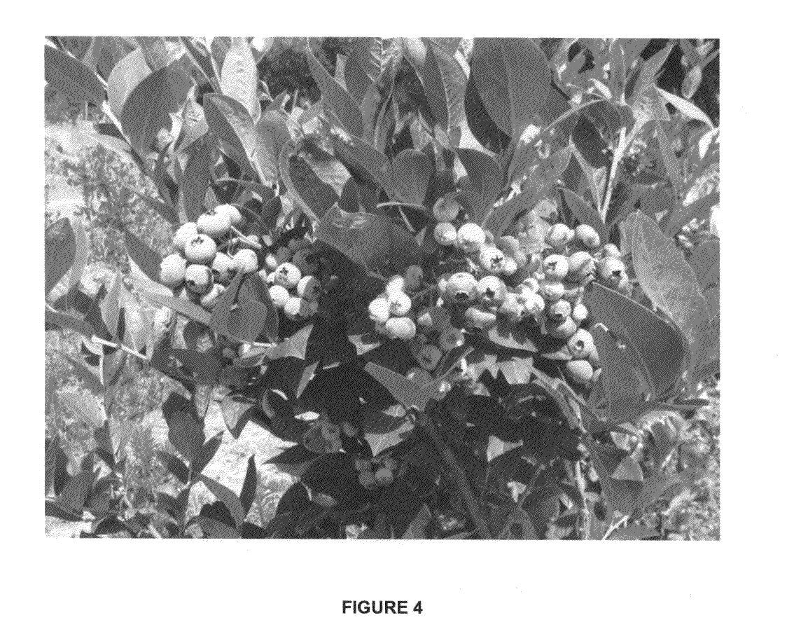

### DETAILED DESCRIPTION

The following is a description of the new variety with color terminology in accordance with The Royal Horticultural Society Colour Charts (R.H.S.C.C.) 2001 edition.

The specimens described were grown in Gilten, Germany. The observations were made in the 2010-2012 seasons on 6-8 year old bushes.
Plant and foliage:
   Plant.—medium size at maturity, managed under standard orchard practice. Growth habit is upright and fruit are borne only on one-year-old shoots.
   Leaves.—mature fully expanded leaves are an average of approximately 78.0 mm long and 37.2 mm wide. The pinnate leaves are oval to oblong in shape with smooth margins. The leaves are coloured near yellow green 146A with a moderate and uniform glossiness on the upper surface and venation coloured near greyed purple 185B on the underside. The petioles are an average of approximately 2.3 mm long.
Fruit:
   Immature fruit.—coloured near yellow green 145C
   Mature fruit.—medium to large in size, firm, with an average fresh weight of 2.2 g and diameter of 18.0 mm. The fruit have a medium to strong bloom with medium blue skin colour after the bloom is removed. The fruit are generally oblate in shape with a small dry scar and an average brix of 14.2%.
   Seeds.—there is an average of between 3 and 6 seeds per fruit with an average length of 1.8 mm, coloured near greyed-orange N170A.
Events: The timing of the beginning of flowering is medium compared to standard V. corymbosum varieties, while the timing of the beginning of fruit ripening is very late—similar to that of ‘Elizabeth’ (not patented).
Pest and disease: ‘Blue Silk’ does not appear particularly resistant or susceptible to any particular pests or diseases known to afflict V. corymbosum.
Hardiness: The plant cold hardiness according to the American zone classification has not been determined. ‘Blue Silk’ has a chilling requirement of between 800-1000 hours.

1. A new and distinct blueberry plant substantially as illustrated and described herein.
